(19) United States
(12) Patent Application Publication
(10) Pub. No.: US 2011/0301380 A1
(43) Pub. Date: Dec. 8, 2011

(54) PROCESS AND APPARATUS FOR PREPARING ISOCYANATES

(75) Inventors: Carsten Knoesche, Niederkirchen (DE); Torsten Mattke, Freinsheim (DE)

(73) Assignee: BASF SE, Ludwigshafen (DE)

(21) Appl. No.: 13/202,175

(22) PCT Filed: Mar. 4, 2010
(86) PCT No.: PCT/EP2010/052748

§ 371 Date: Aug. 18, 2011

(30) Foreign Application Priority Data
Mar. 6, 2009 (EP) 09154490.8

(51) Int. Cl. C07C 263/10; B01J 19/00

(52) U.S. Cl. 560/347; 422/240; 422/198; 422/199; 422/202

(57) ABSTRACT

The invention relates to a process for preparing isocyanates by reacting the corresponding amines with phosgene in the gas phase, optionally in the presence of an inert medium, in which the amine is evaporated in an evaporator to give an amine-comprising gas stream, the phosgene is mixed into the amine-comprising gas stream, and the amine and the phosgene are converted to the isocyanate in a reactor, wherein the temperature of surfaces in contact with the gaseous amine is kept above the dew point limit of the amine-comprising gas stream. The invention further relates to an apparatus for preparing isocyanates by reacting the corresponding amines with phosgene in the gas phase, comprising an evaporator to evaporate the amine and a reactor in which the reaction is effected, and means of connection of evaporator and reactor, wherein surfaces which can come into contact with gaseous amine are provided with a coating which is not wetted by amine or have an averaged roughness depth Rz to DIN ISO 4287 of not more than 10 μm, and/or the apparatus has no dead spaces or thermal bridges.

The invention relates to a process for preparing isocyanates by reacting the corresponding amines with phosgene in the gas phase, optionally in the presence of an inert medium, in which the amine is evaporated in an evaporator and mixed with the phosgene, and the amine and the phosgene are converted to the isocyanate in a reactor.

The preparation of isocyanates by phosgenating the corresponding amines can in principle be effected by a liquid phase or gas phase phosgenation. Gas phase phosgenation is notable in that a higher selectivity, a lower holdup of toxic phosgene and a reduced amount of energy are required.

In gas phase phosgenation, an amine-containing reactant stream and a phosgene-containing reactant stream, each in the gaseous state, are mixed. The amine and the phosgene react with release of hydrogen chloride (HCl) to give the corresponding isocyanates. The amine-containing reactant stream is generally present in the liquid phase and has to be evaporated and optionally superheated before being mixed with the phosgene-containing stream.

Corresponding processes for preparing isocyanates in the gas phase are described, for example, in EP-A 1 319 655, EP-A 1 555 258, EP-A 1 935 876 or EP-A 0 289 840.

In order to mix the amine-containing reactant stream in the gas phase with the phosgene-containing reactant stream, it is necessary first to evaporate the amine-containing reactant stream. A suitable evaporator is disclosed, for example, in EP-A 1 754 698.

However, a disadvantage of the known processes for gas phase phosgenation is that solid deposits form between evaporator and mixing unit, which necessitate cleaning of the system. The cause of this is that the temperature of surfaces in contact with evaporated amine goes below the dew point temperature of the gas mixture locally or over the whole area. Amine precipitates on these surfaces, and, owing to long residence times and high temperatures, cracks gradually and thus forms deposits on the surfaces. These can grow to form coatings or lead to blockages in downstream plant parts, for example a mixing nozzle, as a result of entrainment of solid constituents. Regular cleaning is therefore required. It is necessary for this purpose to shut down the plant. Deposits resulting from condensed and cracked amine can arise, for example, on walls of the evaporator and on downstream pipelines and apparatus, for example droplet separators or heat transferers.

The cause of the temperature going below the dew point limit on the inner surfaces of the amine supply are, for example, heat losses in the pipelines and apparatus, or the addition of colder gaseous inert gas streams. The risk of condensate formation is particularly great especially close to thermal bridges and in dead zones of the gas flow. The dead zones in the gas flow can arise, for example, as a result of measurement ports, for example for pressure measurements or temperature measurements, or as a result of control units such as taps or valves. However, solid deposits also form to an increased extent on rough surfaces or at cracks or scars.

It is therefore an object of the invention to provide a process for preparing isocyanates by reacting the corresponding amines with phosgene in the gas phase, by which occurrence of blockages in the amine supply to the mixing unit are prevented, which necessitate cleaning and hence a shutdown of the plant.

The object is achieved by a process for preparing isocyanates by reacting the corresponding amines with phosgene in the gas phase, optionally in the presence of an inert medium, in which the amine is evaporated in an evaporator to give an amine-comprising gas stream, the phosgene is mixed into the amine-comprising gas stream and the amine and the phosgene are converted to the isocyanate in a reactor, wherein the temperature of surfaces in contact with the gaseous amine is kept above the dew point limit of the amine-comprising gas stream.

Surfaces in contact with the gaseous amine are, for example, surfaces of apparatus, components and pipelines in an apparatus for preparing isocyanates by reacting the corresponding amines with phosgene. Apparatus used is, for example, mixing units, heat transferers or the reactor; components are, for example, valves, taps or measurement and control units.

Suitable measures to keep the temperature of the surfaces in contact with the gaseous amine above the dew point limit of the amine-comprising gas stream are, for example:
(a) superheating the gaseous amine stream in order to rule out local falling below the dew point as a result of heat losses,
(b) insulation of pipelines and apparatus in order to minimize heat losses,
(c) heating of pipelines and apparatus in order to prevent local falling below the dew point,
(d) feeding in an inert gas stream at controlled temperature to increase the dew point of the amine-comprising gas mixture.

The measures can be implemented either individually or in any combination of two or more.

The process according to the invention prevents or significantly reduces the formation of deposits on walls of the plant for preparing isocyanates.

In addition, solid deposits can also be prevented or reduced by construction measures.

The temperature at which the amine condenses out for the amine comprising gas stream at a given pressure is understood as dew point limit or dew point temperature of the amine comprising gas stream, respectively.

A suitable apparatus for preparing isocyanates by reacting the corresponding amines with phosgene in the gas phase comprises an evaporator to evaporate the amine and a reactor in which the reaction is effected, and means of connection of evaporator and reactor. According to the invention, surfaces which can come into contact with gaseous amine are provided with a coating which is not wetted by amine and have an averaged roughness depth Rz to DIN ISO 4287 of not more than 10 μm, and/or the apparatus has no dead spaces or thermal bridges. Use of an inventive apparatus prevents the formation of deposits on plant components which can come into contact with gaseous amine, for example surfaces of the amine evaporator, pipelines which connect the evaporator to the reactor, and on surfaces of the reactor.

Superheating of the amine stream prevents, in the event of heat losses, the temperature from going below the dew point temperature. Given appropriate design, the superheating can be accomplished in the evaporator. Owing to the thermal decomposition of the amines at high temperatures, it is, however, preferred to minimize the superheating of the amine stream. Insulation of pipelines and apparatus allows the heat losses to be minimized and hence the necessary superheating to be kept small. Favorable temperature differences from the condensation limit have been found to be at least 5 K, preferably at least 10 K and more preferably at least 20 K. This especially prevents the local temperature from going below the condensation limit, especially at thermal bridges or in dead zones of the flow.

A further very effective method of preventing occurrence of condensate on the surfaces which are in contact with gaseous amine is that of trace-heating the apparatus and pipelines to ensure a surface temperature of the surfaces which come into contact with gaseous amine above the dew point temperature. Heat losses can be prevented or even compensated for in this way. The trace heating is adjusted so as to establish a surface temperature of at least 5 K, preferably of at least 10 K and more preferably of at least 20 K above the condensation limit. The trace heating of the surfaces can be achieved, for example, by designing walls with a jacket or applying pipe coils through which a heating medium flows or by electrical heating.

Feeding an inert gas at a controlled temperature into the amine stream allows the partial pressure of the amine to be lowered and hence the dew point of the gas mixture to be raised. The inert media used may, for example, be nitrogen, noble gases such as helium or argon, aromatics such as chlorobenzene, o-dichlorobenzene, trichlorobenzene, toluene, xylene, chloronaphthalene, decahydronaphthalene, carbon dioxide, carbon monoxide or mixtures thereof. Preference is given to using nitrogen and/or chlorobenzene. The temperature of the inert gas supplied is adjusted such that a temperature margin of the mixing temperature from the condensation limit of at least 5 K, preferably of at least 10 K and more preferably of at least 20 K exists.

The prevention of the formation of deposits by the process according to the invention allows the service life of the plant for preparing isocyanates to be increased compared to the processes known from the prior art. Less frequent shutdown to clean the plant is necessary.

To prepare the isocyanate, the phosgene and the amine are preferably first fed to a mixing zone in which amine and phosgene are mixed to give a reaction mixture. Subsequently, the reaction mixture is fed to the reactor in which the conversion to the isocyanate is effected. The conversion of amine and phosgene in the reactor proceeds in the gas phase. To this end, the pressure in the reactor is preferably in the range between 0.3 and 5 bar absolute, more preferably in the range from 0.8 to 3.5 bar absolute. The temperature is preferably in the range from 250 to 550° C., especially in the range from 300 to 500° C.

In order to be able to perform the reaction in the gas phase, the amine and the phosgene are added in gaseous form. To this end, the amine preferably has a temperature in the range from 200 to 400° C. The pressure of the amine added is preferably in the range between 0.05 and 3 bar absolute. The temperature of the phosgene added is preferably in the range from 250 to 500° C. To this end, the phosgene is typically heated before addition in the manner known to those skilled in the art.

To heat the phosgene and the amine and to evaporate the amine, for example, electrical heating or direct or indirect heating by combustion of a fuel is used. The fuels used are typically fuel gases, for example natural gas. By virtue of the lowering of the pressure in the course of evaporation and hence the boiling temperature of the amine, however, heating by means of steam is also possible. The pressure of the steam is selected here according to the boiling temperature of the amine. A suitable vapor pressure of the steam is, for example, in the range from 40 to 100 bar. This gives rise to a temperature of the steam in the range from 250 to 311° C.

In general, it is necessary to heat the amine to the reaction temperature in a plurality of stages. In general, the amine, for this purpose, is first preheated, then evaporated and then superheated. In general, the evaporation takes the longest residence times and thus leads to decomposition of the amine. In order to minimize this, evaporation at lower temperatures, as arises, for example, through the lower pressure, is advantageous. In order to superheat the evaporated amine to reaction temperature after the evaporation, heating with steam is generally insufficient. For superheating, electrical heating or direct or indirect heating by combustion of a fuel is therefore typically used.

Owing to the high boiling temperatures of the amine and the resulting large temperature differences from the environment, the walls of plant parts, for example of pipelines which connect the evaporator to the reactor, or of parts of the reactor, for example supply lines or supply nozzles, may have temperatures below the evaporation temperature of the amine. This has the effect that amine can condense out of the gas stream on the walls. Condensed droplets adhere to the wall and lead, owing to the high temperatures, to formation of solids and deposits. Such deposits, for example, reduce pipe cross sections which can, in the case of sufficiently small diameters, in some cases become completely blocked. This necessitates regular cleaning of the plant parts. The process according to the invention reduces or prevents the formation of such deposits, such that higher service lives of the plant can be achieved.

In contrast to the evaporation of the amine, the phosgene is evaporated generally at significantly lower temperatures. For this reason, the phosgene can generally be evaporated using steam. However, the necessary superheating of the phosgene to heat it to reaction temperature is generally also possible only by electrical heating or direct or indirect heating by combustion of a fuel.

The reactor which is used for phosgenation of the amine to prepare isocyanates is known to those skilled in the art. In general, the reactors used are tubular reactors. In the reactor, the amine is reacted with the phosgene to give the corresponding isocyanate and hydrogen chloride. Typically, the phosgene is added in excess, such that the reaction gas which forms in the reactor, as well as the isocyanate formed and the hydrogen chloride, also comprises phosgene.

Amines which can be used to prepare isocyanates are monoamines, diamines, triamines or higher-functionality amines. Preference is given to using monoamines or diamines. According to the amine used, the corresponding monoisocyanates, diisocyanates, triisocyanates or higher-functionality isocyanates are obtained. Preference is given to preparing monoisocyanates or diisocyanates by the process according to the invention.

Diamines and diisocyanates may be aliphatic, cycloaliphatic or aromatic.

Cycloaliphatic isocyanates are those which comprise at least one cycloaliphatic ring system.

Aliphatic isocyanates are those which have exclusively isocyanate groups bonded to straight or branched chains.

Aromatic isocyanates are those which have at least one isocyanate group bonded to at least one aromatic ring system.

The term “(cyclo)aliphatic isocyanates” is used hereinafter for cycloaliphatic and/or aliphatic isocyanates.

Examples of aromatic mono- and diisocyanates are preferably those having 6 to 20 carbon atoms, for example phenyl isocyanate, monomeric 2,4′- and/or 4,4′-methylene-di(phenyl isocyanate) (MDI), 2,4- and/or 2,6-tolylene diisocyanate (TDI) and 1,5- or 1,8-naphthyl diisocyanate (NDI).

Examples of (cyclo)aliphatic diisocyanates are aliphatic diisocyanates such as 1,4-tetramethylene diisocyanate, 1,6-hexamethylene diisocyanate (1,6-diisocyanatohexane), 1,8-octamethylene diisocyanate, 1,10-decamethylene diisocyanate, 1,12-dodecamethylene diisocyanate, 1,14-tetradecamethylene diisocyanate, 1,5-diisocyanatopentane, neopentane diisocyanate, derivatives of lysine diisocyanate, tetramethylxylylene diisocyanate (TMXDI), trimethylhexane diisocyanate or tetramethylhexane diisocyanate, and 3(or 4),8(or 9)-bis(isocyanatomethyl)tricyclo-[5.2.1.0^{2,6}]decane isomer mixtures, and cycloaliphatic diisocyanates such as 1,4-, 1,3- or 1,2-diisocyanatocyclohexane, 4,4′- or 2,4′-di(isocyanatocyclohexyl)methane, 1-isocyanato-3,3,5-trimethyl-5-(isocyanatomethyl)cyclohexane (isophorone diisocyanate), 1,3- or 1,4-bis(isocyanatomethyl)cyclohexane, 2,4- or 2,6-diisocyanato-1-methylcyclohexane.

Preferred (cyclo)aliphatic diisocyanates are 1,6-diisocyanatohexane, 1-isocyanato-3,3,5-trimethyl-5-(isocyanatomethyl)cyclohexane and 4,4′-di(isocyanatocyclohexyl)methane. Particular preference is given to 1,6-diisocyanatohexane, 1-isocyanato-3,3,5-trimethyl-5-(isocyanatomethyl)cyclohexane and 4,4′-di(isocyanatocyclohexyl)methane.

Amines which are used in the process according to the invention for reaction to give the corresponding isocyanates are those in which the amine, the corresponding intermediates and the corresponding isocyanates are present in gaseous form under the selected reaction conditions. Preference is given to amines which decompose over the duration of the reaction under the reaction conditions to an extent of at most 2 mol %, more preferably to an extent of at most 1 mol % and most preferably to an extent of at most 0.5 mol %. Particularly suitable amines here are especially diamines based on (cyclo)aliphatic hydrocarbons having 2 to 18 carbon atoms. Examples thereof are 1,6-diaminohexane, 1,5-diaminopentane, 1,3-bis(aminomethyl)cyclohexane, 1-amino-3,3,5-trimethyl-5-aminomethylcyclohexane (IPDA) and 4,4-diaminodicyclohexylmethane. Preference is given to using 1,6-diaminohexane (HDA).

For the process according to the invention, it is likewise possible to use aromatic amines which can be converted to the gas phase without significant decomposition. Examples of preferred aromatic amines are tolylenediamine (TDA), as the 2,4 or 2,6 isomer or as a mixture thereof, for example as an 80:20 to 65:35 (mol/mol) mixture, diaminobenzene, 2,6-xylidine, naphthyldiamine (NDA) and 2,4′- or 4,4′-methylene(diphenyldiamine) (MDA) or isomer mixtures thereof. Among these preference is given to the diamines, particular preference to 2,4- and/or 2,6-TDA or 2,4′- and/or 4,4′-MDA.

To prepare monoisocyanates, it is likewise possible to use aliphatic, cycloaliphatic or aromatic amines, typically monoamines. A preferred aromatic monoamine is especially aniline.

In the gas phase phosgenation, it is desirable that the compounds which occur in the course of the reaction, i.e. reactants (amine and phosgene), intermediates (especially the carbamoyl and amine hydrochlorides which form as intermediates), end products (diisocyanate), and any inert compounds metered in, remain in the gas phase under the reaction conditions. Should these or other components be deposited from the gas phase, for example on the reactor wall or other apparatus components, these deposits can undesirably alter the heat transfer or the flow through the components affected. This is especially true of occurrence of the amine hydrochlorides which form from free amino groups and hydrogen chloride, since the resulting amine hydrochlorides precipitate readily and are re-evaporable only with difficulty.

In addition to the use of a tubular reactor, it is also possible to use essentially cuboidal reaction chambers, for example plate reactors. Any desired different cross section of the reactor is also possible.

In order to prevent the formation of by-products in the reaction, it is preferred to supply phosgene in excess. In order to supply only the proportion of amines needed for the reaction, it is possible to mix the amine with an inert gas. The proportion of inert gas in the amine can be used to adjust the amount of the amine supplied for a given geometry of the feed orifices for the amine and the phosgene. Inert media which can be added are those which are present in gaseous form in the reaction chamber and do not react with the compounds which occur in the course of the reaction. The inert media used may, for example, be nitrogen, noble gases such as helium or argon, aromatics such as chlorobenzene, o-dichlorobenzene, trichlorobenzene, toluene, xylene, chloronaphthalene, decahydronaphthalene, carbon dioxide or carbon monoxide. Preference is given, however, to using nitrogen and/or chlorobenzene as the inert medium.

Alternatively, it is, however, also possible, for example, in order to avoid too great an excess of phosgene, to add the inert medium to the phosgene.

In general, the inert medium is added in an amount such that the ratio of the gas volumes of inert medium to amine or to phosgene is less than 0.0001 to 30, preferably less than 0.01 to 15 and more preferably less than 0.1 to 5.

Suitable coatings which can prevent adherence of amine which has condensed out are, for example, those which comprise SiOₓ, for example Silcosteel coatings from Restec Corporation.

When the condensation of amine on surfaces is to be prevented by heating the corresponding surfaces, the temperature to which the surface which can come into contact with the amine is heated is preferably at least 5 K, more preferably at least 10 K and especially at least 15 K above the condensation limit of the amine-comprising gas mixture. The condensation limit is determined by the pressure in the amine lines and the inert gas content in the gas mixture.

When, for example, the proportion of amine in the gas stream is increased or the apparatus for preparing isocyanates is operated at a relatively high pressure, it is likewise necessary also to increase the temperatures to which the surfaces which can come into contact with the amine are heated.

The surfaces can be heated by any heating process known to those skilled in the art. Preference is given to heating the surfaces using electrical heating elements. The advantage of the use of electrical heating elements is that they can be set to a given temperature in a simple manner.

In order to reduce or to prevent the formation of undesired by-products, and also to suppress decomposition of the isocyanate formed, the reaction gas is preferably cooled in a quench immediately after the reaction. To this end, a generally liquid quench medium is added. As a result of evaporation of the quench medium, it absorbs heat and leads to rapid cooling of the reaction gas. The addition of the quench medium results in a mixture of reaction gas and quench medium as the product stream.

Rapid cooling is achieved especially by adding the quench medium in finely atomized form. As a result, the quench medium has a large surface area and can rapidly absorb the heat and hence cool the reaction gas.

According to the invention, the quench medium is added in liquid form with a temperature above the condensation temperature of the reaction gas. In order to prevent premature evaporation of the quench medium, it may be necessary to increase the pressure in the feed line compared to the pressure in the quench space. The decompression to the pressure of the quench space can then be achieved through the nozzles themselves or else suitable control units. The decompression of the quench medium and mixing with the hot reaction gases accomplish heating and/or partial or complete evaporation of the quench medium. The heat absorbed in the process leads to cooling of the reaction gases.

Especially in the case of use of a quench medium which has a boiling temperature below the condensation temperature of the reaction gas under the conditions in the quench space, the pressure in the feed lines is elevated compared to the pressure in the quench space in order to prevent evaporation of the quench medium before the addition to the quench space.

The pressure with which the quench medium is added is preferably in the range from 1 to 20 bar, more preferably in the range from 1 to 10 bar and especially in the range from 1 to 8 bar.

The quench medium used for cooling preferably comprises a solvent which is selected from the group consisting of monochlorobenzene, dichlorobenzene, trichlorobenzene, hexane, benzene, 1,3,5-trimethylbenzene, nitrobenzene, anisole, chlorotoluene, o-dichlorobenzene, diethyl isophthalate, tetrahydrofuran, dimethylformamide, xylene, chloronaphthalene, decahydronaphthalene and toluene.

In one embodiment of the invention, the quench may be followed by further stages for cooling the reaction gas. In the individual stages for cooling, the reaction gas is cooled further in each case, until the desired end temperature is attained, with which the reaction gas is sent, for example, to a downstream workup.

In one embodiment, at least one of the stages for cooling the reaction gas which follow the quench is a further quench.

For example, it is possible to scrub the reaction gas leaving the quench and the stages for cooling which may follow with a solvent, preferably at temperatures of more than 130° C. Suitable solvents are, for example, the same substances which can also be used as the quench medium.

In the course of scrubbing, the isocyanate is transferred selectively into the scrubbing solution. Subsequently, the remaining gas and the resulting scrubbing solution are preferably separated by means of rectification into isocyanate, solvent, phosgene and hydrogen chloride.

The gas mixture leaving the reactor is preferably scrubbed in a scrubbing tower, by removing the isocyanate formed from the gaseous gas mixture by condensation in an inert solvent, while excess phosgene, hydrogen chloride and if appropriate the inert medium pass through the workup apparatus in gaseous form. Preference is given to maintaining the temperature of the inert solvent above the dissolution temperature of the carbamoyl chloride corresponding to the amine in the selected scrubbing medium. The temperature of the inert solvent is more preferably maintained above the melting temperature of the carbamoyl chloride corresponding to the amine.

The scrubbing can be performed in any desired apparatus known to those skilled in the art. For example, stirred vessels or other conventional apparatus is suitable, for example columns or mixer-settler apparatus.

The reaction gas leaving the reactor is scrubbed and worked up generally as described, for example, in WO-A 2007/028715.

As already described above, a coating preferably comprises SiOₓ.

In a further preferred embodiment, apparatus for heating the surfaces which can come into contact with gaseous amine is additionally included. Suitable apparatus for heating the surfaces is especially electrical heating elements. However, embodiments using jacket heaters with a corresponding heating medium are also possible. Surfaces which can come into contact with the gaseous amine are, for example, surfaces of the evaporator and of the reactor, and walls of tubes through which the gaseous amine flows. The use of apparatus for heating surfaces makes it possible to keep the surfaces at a temperature above the condensation temperature of the amine-comprising gas mixture, thus making it possible to prevent amine from condensing out. This also prevents deposits from forming on the surfaces.

1. A process for preparing at least one isocyanate, comprising:
a) evaporating an amine in an evaporator to give an amine-comprising gas stream;
b) mixing phosgene with the amine-comprising gas stream; and
c) converting the amine and the phosgene to the isocyanate in a reactor,
wherein:
the amine reacts with the phosgene in a gas phase, optionally in the presence of an inert medium; and
a temperature of at least one surface in contact with the amine-comprising gas stream is kept above a dew point limit of the amine-comprising gas stream.

2. The process of claim 1, wherein the temperature of the at least one surface in contact with the amine-comprising gas stream is kept above the dew point limit of the amine-comprising gas stream by fulfilling at least one of the following:
(a) superheating the amine-comprising gas stream to prevent local temperatures from falling below the dew point limit of the amine-comprising gas stream as a result of heat losses;
(b) insulating at least one pipeline and the apparatus in order to minimize heat losses;
(c) heating the at least one pipelines and the apparatus to prevent temperatures from falling below the dew point limit of the amine-comprising gas stream;
(d) feeding in an inert gas stream at a controlled temperature to increase the dew point of the amine-comprising gas stream.

3. The process of claim 1, wherein the temperature of at least one surface which can come into contact with the amine-comprising gas stream is heated to at least 5 K above a condensation temperature of the amine-comprising gas stream.

4. The process of claim 2, wherein the at least one surface in contact with the amine-comprising gas stream is heated with at least one electrical heating element or at least one jacket heater.

5. The process of claim 2, wherein the feeding in an inert gas stream at a controlled temperature (d) generates a temperature margin of mixing temperature from a condensation limit of the amine-comprising gas stream of at least 5 K.

6. The process of claim 2, wherein the inert gas stream is at least one gas selected from the group consisting of nitrogen, helium, argon, chlorobenzene, o-dichlorobenzene, toluene, xylene, chloronaphthalene, decahydronaphthalene, carbon dioxide, and carbon monoxide.

7. The process of claim 1, wherein a reaction gas comprising the isocyanate and hydrogen chloride leaves the reactor and is cooled in a quench by adding a liquid quench medium, which forms a mixture of the reaction gas and the liquid quench medium as a product stream.

8. The process of claim 7, wherein the liquid quench medium comprises at least one solvent selected from the group consisting of monochlorobenzene, dichlorobenzene, trichlorobenzene, hexane, benzene, 1,3,5-trimethylbenzene, nitrobenzene, anisole, chlorotoluene, o-dichlorobenzene, diethyl isophthalate, tetrahydrofuran, dimethylformamide, xylene, chloronaphthalene, decahydronaphthalene and toluene.

9. The process claim 7, wherein the quench is followed by at least one further stage of workup of the product stream.

10. An apparatus for preparing isocyanates, comprising:
an evaporator to evaporate an amine;
a reactor, wherein the amine reacts with phosgene in the gas phase; and
a connection of the evaporator and the reactor,
wherein:
at least one surface which can come into contact with a gaseous amine is provided with a coating which is not wetted by the amine and have an averaged roughness depth Rz to DIN ISO 4287 of not more than 10 μm; and
the apparatus has no dead spaces or thermal bridges.

11. The apparatus of claim 10, wherein the coating comprises SiOₓ.

12. The apparatus of claim 10, wherein an apparatus for heating the surfaces which can come into contact with the gaseous amine is included.

13. The process of claim 1, wherein the amine reacts with the phosgene in the presence of an inert medium.

14. The process of claim 3, wherein the at least one surface in contact with the amine-comprising gas stream is heated with at least one electrical heating element or at least one jacket heater.

15. An apparatus for preparing isocyanates, comprising:
an evaporator to evaporate an amine;
a reactor, wherein the amine reacts with phosgene in the gas phase; and
a connection of the evaporator and the reactor,
wherein:
at least one surface which can come into contact with a gaseous amine is provided with a coating which is not wetted by the amine and have an averaged roughness depth Rz to DIN ISO 4287 of not more than 10 μm; or
the apparatus has no dead spaces or thermal bridges.
